(19) United States
(12) Patent Application Publication
(10) Pub. No.: US 2014/0349828 A1
(43) Pub. Date: Nov. 27, 2014

(54) APPARATUS, SYSTEM, AND METHOD FOR COLLECTING A TARGET MATERIAL

(71) Applicant: RareCyte, Inc., Seattle, WA (US)

(72) Inventors: Lance U'ren, Seattle, WA (US); Joshua Nordberg, Bainbridge Island, WA (US); Daniel Campton, Seattle, WA (US); Steve Quarre, Woodinville, WA (US); Ronald Seubert, Sammamish, WA (US); David Stewart, Seattle, WA (US); Jennifer Chow, Seattle, WA (US); Jonathan Lundt, Seattle, WA (US)

(73) Assignee: RareCyte, Inc., Seattle, WA (US)

(21) Appl. No.: 14/266,939

(22) Filed: May 1, 2014

Related U.S. Application Data
(60) Provisional application No. 61/818,301, filed on May 1, 2013.
(60) Provisional application No. 61/869,866, filed on Aug. 26, 2013.
(60) Provisional application No. 61/935,457, filed on Feb. 4, 2014.

(51) Int. Cl. A61M 1/36

(52) U.S. Cl. CPC A61M 1/3693
USPC 494/37

(57) ABSTRACT

This disclosure is directed to an apparatus, system and method for retrieving a target material from a sample. A fraction-density-altering solution may be added to a vessel that contains the sample to change the density of a first fraction of the sample without changing the density of the target material or the density of any other sample fraction. A collector may be inserted into the vessel to funnel the target material from the sample into the collector or into a processing receptacle included in the collector. In one implementation, the collector may include a cannula which extends into a chamber at a first end of the collector and a funnel at a second end that that is in fluid communication with the cannula. The chamber is sized and shaped to hold the processing receptacle. In another implementation, the processing receptacle may be inserted into a bore within the collector.

### CROSS-REFERENCE TO A RELATED APPLICATION

This application claims the benefit of Provisional Application No. Provisional Application No. 61/818,301, filed May 1, 2013, Provisional Application No. 61/869,866, filed Aug. 26, 2013, and Provisional Application No. 61/935,457, filed Feb. 4, 2014.

### TECHNICAL FIELD

This disclosure relates generally to density-based fluid separation and, in particular, to retrieving a target material from a suspension.

### BACKGROUND

Suspensions often include materials of interests that are difficult to detect, extract and isolate for analysis. For instance, whole blood is a suspension of materials in a fluid. The materials include billions of red and white blood cells and platelets in a proteinaceous fluid called plasma. Whole blood is routinely examined for the presence of abnormal organisms or cells, such as ova, fetal cells, endothelial cells, parasites, bacteria, and inflammatory cells, and viruses, including HIV, cytomegalovirus, hepatitis C virus, and Epstein-Barr virus. Currently, practitioners, researchers, and those working with blood samples try to separate, isolate, and extract certain components of a peripheral blood sample for examination. Typical techniques used to analyze a blood sample include the steps of smearing a film of blood on a slide and staining the film in a way that enables certain components to be examined by bright field microscopy.

On the other hand, materials of interest that occur in a suspension with very low concentrations are especially difficult if not impossible to detect and analyze using many existing techniques. Consider, for instance, circulating tumor cells (“CTCs”), which are cancer cells that have detached from a tumor, circulate in the bloodstream, and may be regarded as seeds for subsequent growth of additional tumors (i.e., metastasis) in different tissues. The ability to accurately detect and analyze CTCs is of particular interest to oncologists and cancer researchers. However, CTCs occur in very low numbers in peripheral whole blood samples. For instance, a 7.5 ml sample of peripheral whole blood sample that contains as few as 5 CTCs is considered clinically relevant for the diagnosis and treatment of a cancer patient. In other words, detecting 5 CTCs in a 7.5 ml blood sample is equivalent to detecting 1 CTC in a background of about 40-60 billion red and white blood cells, which is extremely time consuming, costly and difficult to accomplish using blood film analysis.

As a result, practitioners, researchers, and those working with suspensions continue to seek systems and methods for accurate analysis of suspensions for the presence or absence rare materials of interest.

### DESCRIPTION OF THE DRAWINGS

FIGS. 1A-1B an example collector.

FIGS. 1C-1D an example collector.

FIGS. 2A-2C show example collectors.

FIGS. 3A-3B show an example collector and processing receptacle system.

FIG. 3C shows an example collector and processing receptacle system.

FIG. 4 shows an example collector, processing receptacle, and vessel system.

FIG. 5 shows a flow diagram of an example method for retrieving a target material.

FIG. 6 shows an example insert within an example vessel.

FIG. 7 shows a flow diagram of an example method for retrieving a target material.

FIG. 8 shows a flow diagram of an example method for retrieving a target material.

### DETAILED DESCRIPTION

This disclosure is directed to an apparatus, system and method for retrieving a target material from a sample. A fraction-density-altering solution may be added to a vessel that contains the sample to change the density of a first fraction of the sample without changing the density of the target material or the density of any other sample fraction. A collector may be inserted into the vessel to funnel the target material from the sample into the collector or into a processing receptacle included in the collector. In one implementation, the collector may include a cannula which extends into a chamber at a first end of the collector and a funnel at a second end that that is in fluid communication with the cannula. The chamber is sized and shaped to hold the processing receptacle. In another implementation, the processing receptacle may be inserted into a bore within the collector.

### Collector

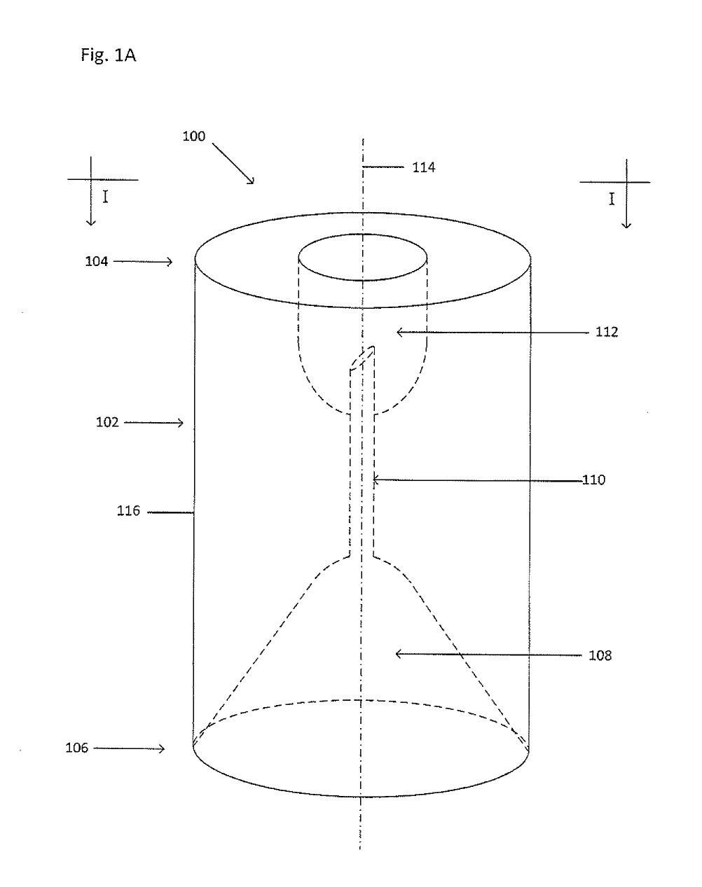

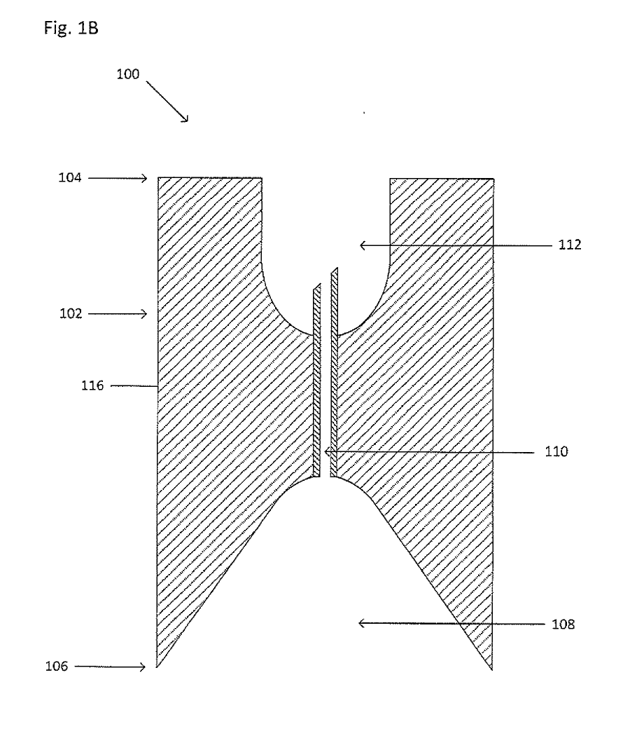

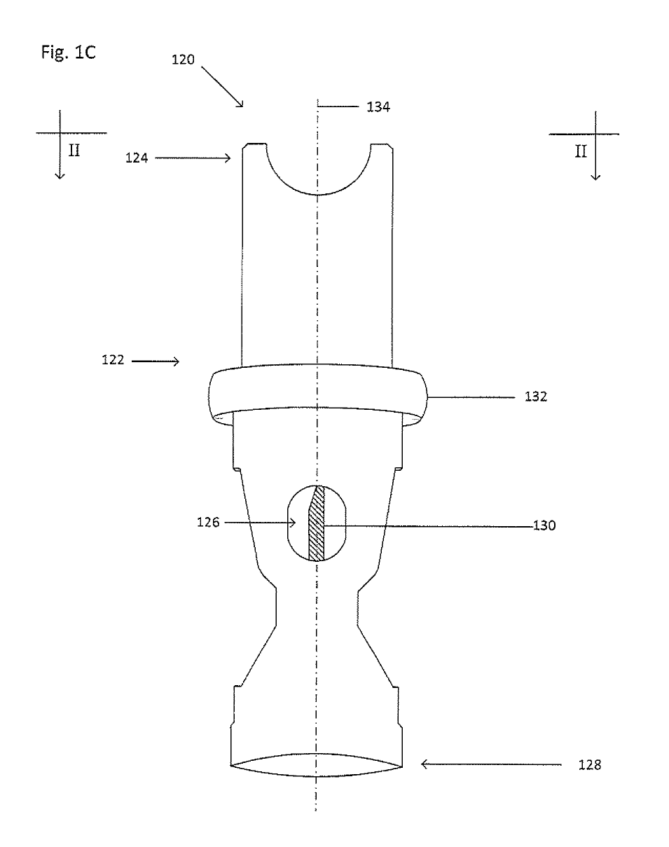

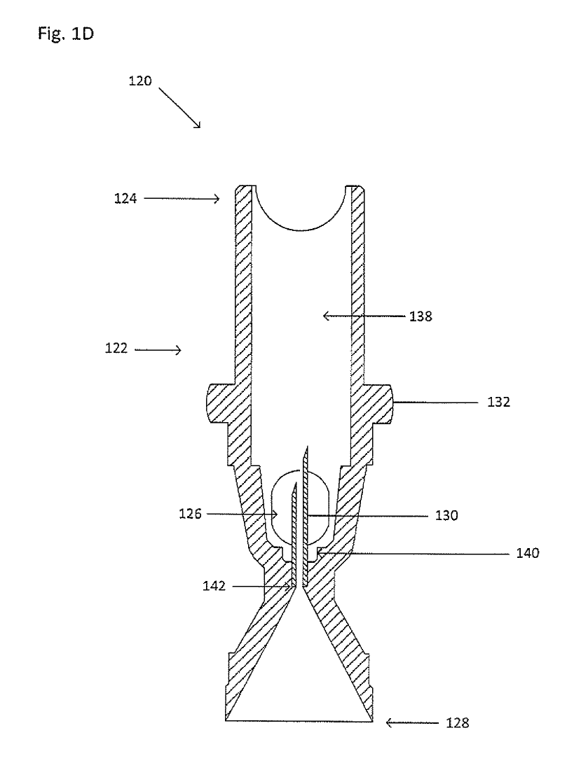

FIG. 1A shows an isometric view of a collector 100. FIG. 1B shows a cross-section view of the collector 100 taken along the line I-I. Dot-dashed line 114 represents the central or highest-symmetry axis of the collector 100. The collector 100 may be sized and shaped to fit within a vessel containing or capable of holding a suspension, the suspension suspected of including a target material. The collector 100 may engage in an interference or sealed fit with an inner wall of the vessel to inhibit any portion of the suspension from being located between the inner wall of the vessel and an outer wall 116 of the collector 100. The collector 100 funnels the target material from the suspension through a cannula 110 and into a processing receptacle (not shown) located within a chamber 112.

The collector 100 includes a primary body 102. The primary body 102 includes a first end 104 and a second end 106. The primary body 102 may be any appropriate shape, including, but not limited to, cylindrical, triangular, square, rectangular, or the like. The collector 100 also includes a funnel 108 which is a concave cavity extending from the second end 106 into the primary body 102. The funnel 108 channels a target material from below the second end 106 into a cannula 110 which is connected to and in fluid communication with a apex of the funnel 108. The apex of the funnel 108 has a smaller diameter than the mouth of the funnel 108. The funnel 108 is formed by a tapered wall that may be straight, curvilinear, arcuate, or the like.

The cannula 110, such as a tube or a needle, including, but not limited to a non-coring needle, extends from the apex of the funnel 108 and into the chamber 112. The chamber 112 is a cavity to accept and support the processing receptacle (not shown). The chamber 112 may be threaded to engage a threaded portion of the processing receptacle (not shown). The cannula 110 may extend any appropriate distance into the chamber 112 in order to puncture or be inserted into the processing receptacle (not shown). The cannula 110 may include a flat tip or a tapered tip. The chamber 112 is a concave cavity extending from the first end 104 into the primary body 102. The chamber 112 may be any appropriate depth to accept and support the processing receptacle (not shown). Furthermore, the chamber 112 may be any appropriate shape, including, but not limited to, semi-spherical, conical, pyramidal, or the like.

The collector 100 may also include a retainer (not shown) to prevent the collector 100 from sliding relative to the vessel, thereby keeping the collector 100 at a pre-determined height within the vessel. The retainer (not shown) may be a shoulder extending radially from the first end 104, a clip, a circular protrusion that extends beyond the circumference of the cylindrical primary body 102, a detent, or the like.

FIG. 1C shows an isometric view of a collector 120. FIG. 1D shows a cross-section view of the collector 120 taken along the line II-II. Dot-dashed line 134 represents the central or highest-symmetry axis of the collector 120. The collector 120 is similar to the collector 100, except that the collector 120 includes a window 126, a ridge 132, and a primary body 122 to accommodate a greater portion of the processing receptacle (not shown). In the collector 100 shown in FIGS. 1A-1B with collector 120 shown in FIGS. 1C-1D, the primary body 122 is longer than the primary body 102. The window 126 permits an operator to confirm proper placement and chambering of the processing receptacle (not shown) within the collector 120. The collector 120 includes a cavity 138 dimensioned to accept and hold at least a portion of the processing receptacle (not shown). The cavity 138 may have a tapered or stepped bottom end 140 to act as a chamber on which the processing receptacle (not shown) may rest. The first end 124 may include cut-outs to permit proper grip of the processing receptacle (not shown) for insertion and removal. A second end 128 forms an interference or sealed fit with the vessel to prevent fluid from flowing around the collector 120. The collector 120 also includes a shelf 142 to support a cannula 130 and provide a seamless fluid transition between the apex of the funnel and the bottom end of the cannula 130. The collector 120 also includes a ridge 132, which extends circumferentially around the primary body 122. The ridge 132 may be larger than the inner diameter of the vessel so as to rest on the open end of the vessel and, upon applying a lock ring (not shown) to the outside of the vessel and the ridge 132, to inhibit movement of the collector 120 relative to the vessel. The lock ring (not shown) applies pressure to the vessel along the ridge 132. The lock ring may be a two-piece ring, a one piece ring wrapping around the full circumference of the vessel, or a one piece ring wrapping around less than the full circumference of the vessel, such as one-half (½), five-eighths (⅝), two-thirds (⅔), three-quarters (¾), seven-eighths (⅞), or the like. Alternatively, the ridge 132 may fit within the vessel.

The primary body may be composed of a variety of different materials including, but not limited to, a ceramic; metals; organic or inorganic materials; and plastic materials, such as polyoxymethylene (“Delrin®”), polystyrene, acrylonitrile butadiene styrene (“ABS”) copolymers, aromatic polycarbonates, aromatic polyesters, carboxymethylcellulose, ethyl cellulose, ethylene vinyl acetate copolymers, nylon, polyacetals, polyacetates, polyacrylonitrile and other nitrile resins, polyacrylonitrile-vinyl chloride copolymer, polyamides, aromatic polyamides (“aramids”), polyamide-imide, polyarylates, polyarylene oxides, polyarylene sulfides, polyarylsulfones, polybenzimidazole, polybutylene terephthalate, polycarbonates, polyester, polyester imides, polyether sulfones, polyetherimides, polyetherketones, polyetheretherketones, polyethylene terephthalate, polyimides, polymethacrylate, polyolefins (e.g., polyethylene, polypropylene), polyallomers, polyoxadiazole, polyparaxylene, polyphenylene oxides (PPO), modified PPOs, polystyrene, polysulfone, fluorine containing polymer such as polytetrafluoroethylene, polyurethane, polyvinyl acetate, polyvinyl alcohol, polyvinyl halides such as polyvinyl chloride, polyvinyl chloride-vinyl acetate copolymer, polyvinyl pyrrolidone, polyvinylidene chloride, specialty polymers, polystyrene, polycarbonate, polypropylene, acrylonitrite butadiene-styrene copolymer, butyl rubber, ethylene propylene diene monomer; and combinations thereof.

The cannula may be composed of a variety of different materials including, but not limited to, a ceramic; metals; organic or inorganic materials; and plastic materials, such as a polypropylene, acrylic, polycarbonate, or the like; and combinations thereof. The cannula may have a tip along a longitudinal axis of the cannula.

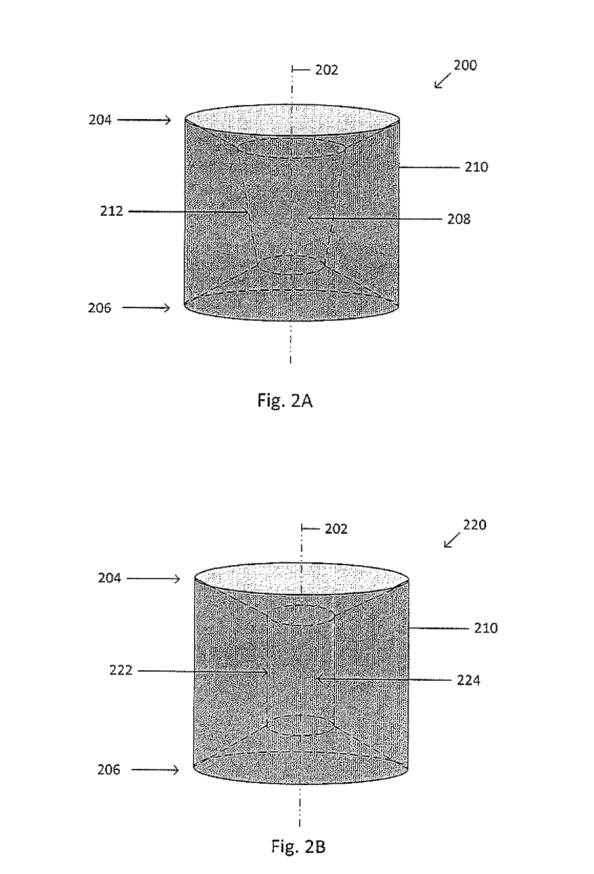

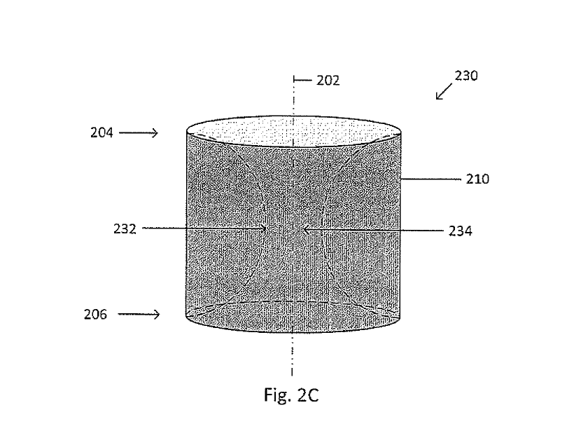

FIG. 2A shows an isometric view of a collector 200. Dot-dashed line 202 represents the central or highest-symmetry axis of the collector 200. The collector 200 includes a first side 204, a second side 206, and a bore 208. An inner wall 212 to form the bore 208, 224, and 234 may be tapered (i.e. becoming narrower from the first side 204 to the second side 206; or becoming wider from the first side 204 to the second 206), as shown in FIG. 2A.

The first and second sides 204 and 206 may be connected to the inner wall 212 via straight walls (i.e. first and second sides 204 and 206 are planar), tapered walls, or at least partially arcuate walls.

The collector 200 may be sized and shaped to fit within a vessel containing or capable of holding a suspension. The collector 200 fits against an inner wall of the vessel, such that no portion of the suspension may be located between the inner wall of the vessel and the outer wall 210 of the collector 200. The collector 200 gathers a sample within the bore 208. The bore 208 may be expandable, such that the diameter of the bore 208 may increase during centrifugation and then return to a resting diameter when not under centrifugation. Expanding the diameter may allow for less constricted flow of fluid and suspension components during centrifugation. The collector 200 may be composed of a ceramic, metal, polymer, flexible polymer, glass, organic or inorganic materials, or the like.

FIG. 2B shows an isometric view of a collector 220. The collector 220 is similar to the collector 200, except that an inner wall 222 of the collector 220 may be straight (i.e. having a uniform diameter from the first side 204 to the second side 206). FIG. 2C shows an isometric view of a collector 230. The collector 230 is similar to the collector 200, except that an inner wall 232 of the collector 230 may be at least partially arcuate (i.e. concave, convex, or curvilinear).

The collector may also include a filter (not shown). The filter (not shown) may be located at the second side or in the bore. The filter (not shown) is configured to provide a more pure sample by permitting a target material to pass through, while inhibiting non-target material from passing through.

### Collection System

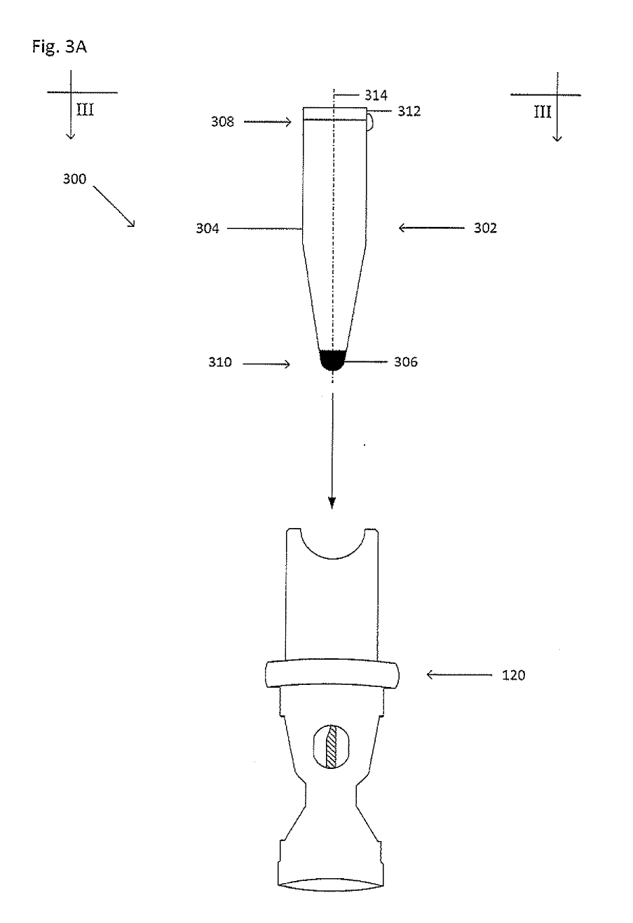

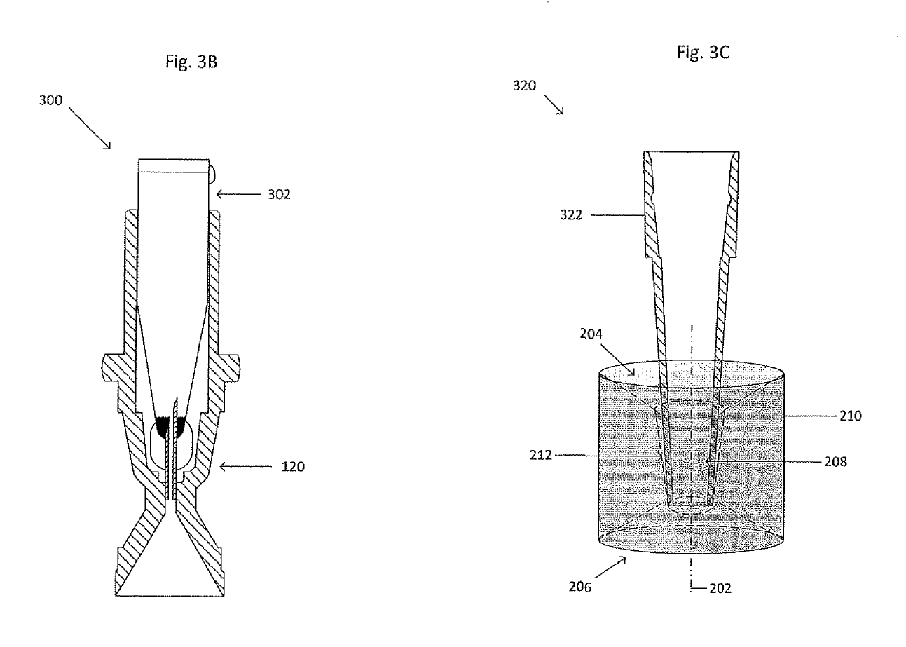

FIG. 3A shows an exploded view of an example collection system 300 including the collector 120 and a processing receptacle 302. FIG. 3B shows a cross-sectional view of the processing receptacle inserted into the chamber 138 of the collector 120 and processing receptacle system 302 taken along the line III-III shown in FIG. 3A. The processing receptacle 302 to receive a target material or a portion of a target material, may be an Eppendorf tube, a syringe, a needle, a pipet tip, a test tube, or the like. The processing receptacle may have a closed end 310 and an open end 308. The open end 308 is sized to receive a cap 312. The cap 312 may be composed of re-sealable rubber or other suitable re-sealable material that may be repeatedly punctured with a needle or other sharp implement to access the contents stored in the processing receptacle 302 interior and re-seals when the needle or implement is removed. Alternatively, the processing receptacle 302 may also have two open ends that are sized to receive caps. Alternatively, the processing receptacle 302 may have two closed ends. The processing receptacle 302 may have a tapered geometry that widens or narrows toward the open end 308; the processing receptacle 302 may have a generally cylindrical geometry; or, the processing receptacle 302 may have a generally cylindrical geometry in a first segment and a cone-shaped geometry in a second segment, where the first and second segments are connected and continuous with each other. Although at least one segment of the processing receptacle 302 has a circular cross-section, in other embodiments, the at least one segment may have elliptical, square, triangular, rectangular, octagonal, or any other suitable cross-sectional shape. The processing receptacle 302 may be composed of a transparent, semitransparent, opaque, or translucent material, such as plastic or another suitable material. The processing receptacle includes a central axis 314. The processing receptacle 302 may also include a plug 306 at the closed end 310 to permit the introduction of the target material or to exchange the target material with a displacement fluid. The closed end 310 may be threaded to provide for a threaded connection with a threaded chamber 138 of the collector 120. The processing receptacle 302 may be composed of glass, plastic, or other suitable material.

The plug 306 may be composed of re-sealable rubber or other suitable re-sealable material that may be repeatedly punctured with a needle or other sharp implement to access the contents of the processing receptacle 302 interior or permit introduction of contents into the processing receptacle 302 and re-seals when the needle or implement is removed. The plug 306 may be formed in the closed end 310 of the processing receptacle 302 using heated liquid rubber that may be shaped and hardens as the rubber cools. The adhesive used to attach a plug to the wall may be a polymer-based adhesive, an epoxy, a contact adhesive or any other suitable material for bonding or creating a thermal bond. Alternatively, the plug 306 may be injected into the processing receptacle 302. Alternatively, the plug 306 may be pre-molded and then inserted into the processing receptacle 302.

When the cannula 130 includes a tapered tip, a portion of the tapered tip may extend into an inner cavity of the processing receptacle 302, whereas another portion of the tapered tip may not enter the inner cavity of the processing receptacle 302. The inner cavity of the processing receptacle 302 is the portion of the processing receptacle 302 to hold the suspension. The cannula 130 may be covered by a resealable sleeve (not shown) to prevent the target material from flowing out unless the processing receptacle 302 is in the chamber 138 and is inserted to a depth appropriate enough for the cannula 130 to penetrate the processing receptacle 302. The resealable sleeve (not shown) covers the cannula 130, is spring-resilient, may be penetrated by the cannula 130, and is made of an elastomeric material capable of withstanding repeated punctures while still maintaining a seal.

The processing receptacle 302 may include a flexible cap that may be pushed to dispense a pre-determined volume therefrom and onto the substrate. The cap 312 may be flexible or the cap 312 may be removed and the flexible cap inserted into the open end 308. Alternatively, the processing receptacle 302 may be attached to (i.e. after accumulating the target material) or may include a dispenser, which is capable of dispensing a pre-determined volume of target material from the processing receptacle 302 onto another substrate, such as a microscope slide. The dispenser may repeatedly puncture the re-sealable cap 312 or compress the material within the processing receptacle 302 to withdraw and dispense the pre-determined volume of target material onto the substrate. Alternatively, the cap 312 may be removed and the dispenser (not shown) may be inserted directly into the processing receptacle 302 to dispense the sample-processing solution mixture.

FIG. 3C shows an example collection system 320 including the collector 200 and a processing receptacle 322. The processing receptacle 322 is similar to the processing receptacle 302. The processing receptacle 322 may be inserted into the collector 200. The processing receptacle 322 may receive the target material or the portion of the target material. Furthermore, the processing receptacle 322 may be removed from the collector 200 and then placed into another vessel, such as a tube, an Eppendorf tube or a slide, to transfer the target material or the portion of the target material to the other vessel, such as by centrifugation, for further processing. Alternatively, an adapter, such as a ferrule, may be included to connect the processing receptacle 322 to the collector 100. The adapter may be metal, plastic, glass, or the like.

### Collection System and Vessel

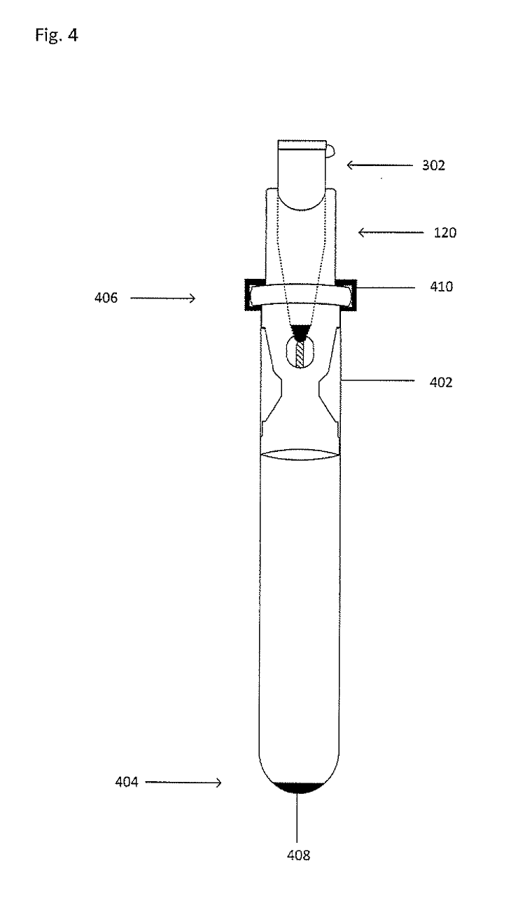

FIG. 4 shows an isometric view of the example collection system 300 within a vessel 402. In the example of FIG. 4, the vessel 402 has a circular cross-section, a first closed end 404, and a second open end 406. The open end 406 is sized to receive the collector 120. The collector 120 may be held in place by a lock ring 410. The vessel may also have two open ends that are sized to receive collectors. The vessel 402 has a generally cylindrical geometry, but may also have a tapered geometry that widens, narrows, or a combination thereof towards the open end 406. Although the vessel 402 has a circular cross-section, in other embodiments, the vessel 402 may have elliptical, square, triangular, rectangular, octagonal, or any other suitable cross-sectional shape that substantially extends the length of the tube. The vessel 402 may also include a septum 408 at the closed end 404 to permit the introduction of a fluid, such as by a needle, or to permit the removal of a fluid, the sample, or a sample fraction with a syringe, a needle, by draining, or the like.

### Methods

For the sake of convenience, the methods are described with reference to an example suspension of anticoagulated whole blood. But the methods described below are not intended to be so limited in their scope of application. The methods, in practice, may be used with any kind of sample, such as a suspension or other biological fluid. For example, a sample may be urine, blood, bone marrow, cystic fluid, ascites fluid, stool, semen, cerebrospinal fluid, synovial fluid, nipple aspirate fluid, saliva, amniotic fluid, vaginal secretions, mucus membrane secretions, aqueous humor, vitreous humor, vomit, a suspension derived from a tissue sample or a culture sample, and any other physiological fluid or semi-solid. It should also be understood that a target material may be a fraction of a sample or a sub-fraction of a fraction, such as a portion of buffy coat. The target material may include an analyte, such as a cell, such as ova, a nucleated red blood cell, or a circulating tumor cell (“CTC”), a circulating endothelial cell, a fetal cell, a vesicle, a liposome, a protein, a nucleic acid, a biological molecule, a naturally occurring or artificially prepared microscopic unit having an enclosed membrane, a parasite (e.g. spirochetes, such as Borrelia burgdorferi), a microorganism, a virus, or an inflammatory cell; or, the target material may be the analytes.

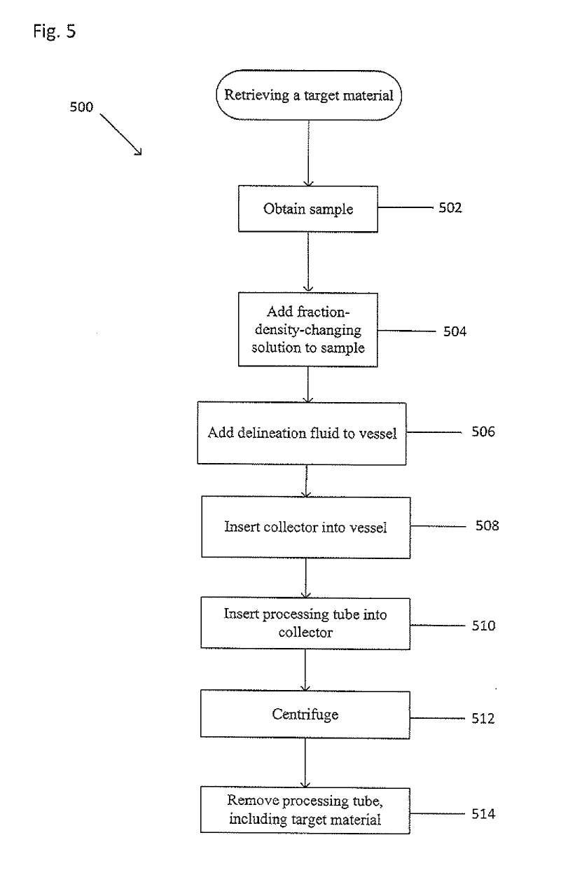

FIG. 5 shows an example method 500 for retrieving a target material. In block 502, a sample, such as anticoagulated whole blood, may be obtained. Suppose, for example, the whole blood includes three fractions. For convenience sake, the three fractions include plasma, buffy coat, and red blood cells. Typically, plasma is the least dense, red blood cells are the densest, and the buffy coat has a density in between the plasma and the red blood cells. The whole blood may be added to a vessel or collected directly into the vessel, such as a test tube or a collection tube (e.g. blood collection tube). The vessel may include a septum in a closed end to permit the removal of a fluid, the sample, or a sample fraction with a syringe, by draining, or the like. The vessel containing the sample may undergo a pre-spin to separate the sample into fractions and to remove at least a portion of one fraction. In block 504, a fraction-density-changing solution, such as a plasma-density-changing solution, may be added to the blood sample to change the density of the plasma relative to the densities of the buffy and the red blood cells. The whole blood and fraction-density-changing solution may then undergo centrifugation.

In one instance, the density of the plasma may be changed to be greater than the density of the buffy coat but less than the density of the red blood cells. When the density of the plasma is changed to be greater than the density of the buffy coat but less than the density of the red blood cells, the hematocrit of the blood may be obtained to calculate the amount of plasma-density-changing solution to be added to the blood sample based on the amount of plasma within the blood sample. In another instance, the density of the plasma may be changed to be greater than the densities of both the buffy coat and the red blood cells. Alternatively, more than one fraction-density-changing solution may be added to change the density of another non-target material, such as the red blood cells, to effect a greater demarcation between the target material and the non-target materials. The fraction-density-changing solution may be isotonic or non-isotonic, so as to alter the density of a target material or a non-target material.

The fraction-density-altering solution may be miscible with the suspension fluid and inert with respect to the suspension materials. Examples of suitable fraction-changing-density solutions include, but are not limited to, solution of colloidal silica particles coated with polyvinylpyrrolidone (e.g. Percoll), polysaccharide solution (e.g. Ficoll), iodixanol (e.g. OptiPrep), a complex, branch glucan (e.g. Dextran), cesium chloride, sucrose, sugar-based solutions, polymer solutions, and multi-phase polymer solutions.

In block 506, a delineation fluid may be added to the vessel. The delineation fluid may be layered on top of the blood sample. It may be desirous to gently layer the delineation fluid on top of the blood sample to inhibit mixing of the delineation fluid with the blood sample. The delineation fluid may be used to cause further separation between the target material and any non-target material below the target material after centrifugation, for example, to further separate the buffy coat and the plasma when the density of the plasma is between the densities of the buffy coat and the red blood cells, or to further separate the buffy coat from the red blood cells when the density of the red blood cells is between the densities of the buffy coat and the plasma. The delineation fluid may lyse or congeal a non-target material, such as red blood cells of a whole blood sample. The delineation fluid may be isotonic or non-isotonic, so as to alter the density of a target material or a non-target material.

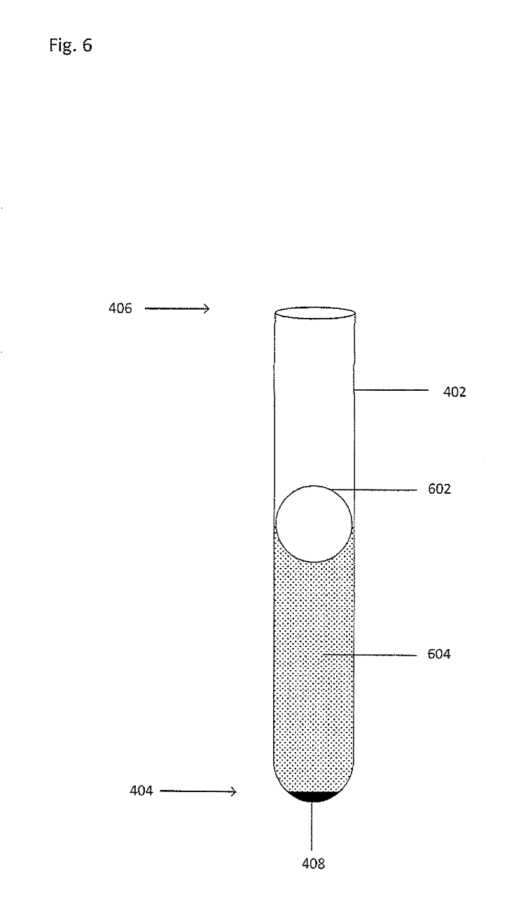

FIG. 6 shows an insert 602 that may be added to the vessel 402, prior to introducing the delineation fluid into the vessel 402, to control the addition of the delineation fluid and inhibit mixing with a whole blood sample-and-fraction-density-altering solution mixture 604. The insert 602 may have a density less than the whole sample-and-fraction-changing-density solution mixture 604. The density of the insert 602 may be greater than the buffy coat and the delineation fluid but less than the density of the red blood cells and the changed density of the plasma. The insert 602 may be a float having any appropriate shape or geometry, a sphere, a disc, a disc with holes, a valve, a valve with a collar, a diffusing membrane, a cylinder, or the like.

The delineation fluid may be miscible or immiscible with the suspension fluid and inert with respect to the suspension materials. Examples of delineation fluids include, but are not limited to, solution of colloidal silica particles coated with polyvinylpyrrolidone (e.g. Percoll), polysaccharide solution (e.g. Ficoll), iodixanol (e.g. OptiPrep), a complex, branch glucan (e.g. Dextran), cesium chloride, sucrose, sugar-based solutions, polymer-based solutions, surfactants, an organic solvent, an oil, olive oil, mineral oil, silicone oil, silicon and silicon-based liquids, such as phenylmethyl siloxane.

Returning to FIG. 5, in block 508, a collector may be inserted into the vessel. The collector may be inserted at least partially into the delineation fluid. A waste vessel may be inserted into the collector so as to remove any non-target material (i.e. fluid) from the cannula. The waste vessel may then be removed and discarded. The waste vessel may be an empty processing receptacle, similar to that shown in FIGS. 3A-3B. Alternatively, the waste vessel may be an empty processing receptacle similar to the processing receptacle shown in FIGS. 3A-3B, except that instead of the plug, a needle or cannula may extend out from the closed end to be inserted into the cannula of the collector. Alternatively, the collector may be inserted into the vessel before the delineation fluid. The delineation fluid may be introduced through the collector.

Returning to FIG. 5, in block 510, a processing receptacle including a displacement fluid may then be added to the collector. Alternatively, the processing receptacle may be inserted into the collector prior to the collector being inserted the vessel. In block 512, the vessel, the collector, and the processing receptacle are centrifuged. During centrifugation, the blood sample separates into its respective fractions based on density, the huffy coat moves above the delineation fluid, and the displacement fluid moves from the processing receptacle into the vessel via the collector and displaces the buffy coat from the vessel into the processing receptacle via the collector. In block 514, the processing receptacle, now including the buffy coat, is removed from the collector.

The displacement fluid may be miscible or immiscible with the suspension fluid and inert with respect to the suspension materials. The displacement fluid 208 has a greater density than the density of the target material of the suspension (the density may be less than the density of at least one other suspension fraction or the density may be greater than all of the suspension fractions) and is inert with respect to the suspension materials. Examples of suitable displacement fluids include, but are not limited to, solution of colloidal silica particles coated with polyvinylpyrrolidone (e.g. Percoll), polysaccharide solution (e.g. Ficoll), iodixanol (e.g. OptiPrep), a complex, branch glucan (e.g. Dextran), cesium chloride, sucrose, sugar-based solutions, polymer-based solutions, surfactants, an organic solvent, a liquid wax, an oil, olive oil, mineral oil, silicone oil, and ionic liquids; perfluoroketones, such as perfluorocyclopentanone and perfluorocyclohexanone, fluorinated ketones, hydrofluoroethers, hydrofluorocarbons, perfluorocarbons, perfluoropolyethers, silicon and silicon-based liquids, such as phenylmethyl siloxane.

The processing receptacle 302 may also include a processing solution to effect a transformation on the target material when the target material enters the processing receptacle 302. The processing solution may be a preservative, a fixative, a cell adhesion solution, a dye, a freezing stabilization media, or the like. Unlike the displacement fluid, most, if not all, of the processing solution remains within the processing receptacle 302 upon centrifugation, thereby effecting the transformation on the target material in one manner or another (i.e. preserving, fixing, increasing adhesion properties, or the like) in the processing receptacle 302. The processing solution may be introduced as a liquid or as a liquid container in a casing. The casing may be dissolvable in an aqueous solution but not in the displacement fluid (such as a gel cap); or, the casing may be breakable, such that the casing breaks when the processing receptacle 302 is shaken in a vortex mixer. Additionally, more than one processing solution may be used.

Furthermore, when the vessel includes a septum in the closed end, the plasma, for example, may be removed through the septum with a needle, syringe, by draining, or the like. The plasma may then be further processed and analyzed.

Sequential density fractionation is the division of a sample into fractions or of a fraction of a sample into sub-fractions by a step-wise or sequential process, such that each step or sequence results in the collection or separation of a different fraction or sub-fraction from the preceding and successive steps or sequences. In other words, sequential density fractionation provides individual sub-populations of a population or individual sub-sub-populations of a sub-population of a population through a series of steps. For example, buffy coat is a fraction of a whole blood sample. The buffy coat fraction may be further broken down into sub-fractions including, but not limited to, reticulocytes, granulocytes, lymphocytes/monocytes, and platelets. These sub-fractions may be obtained individually by performing sequential density fractionation.

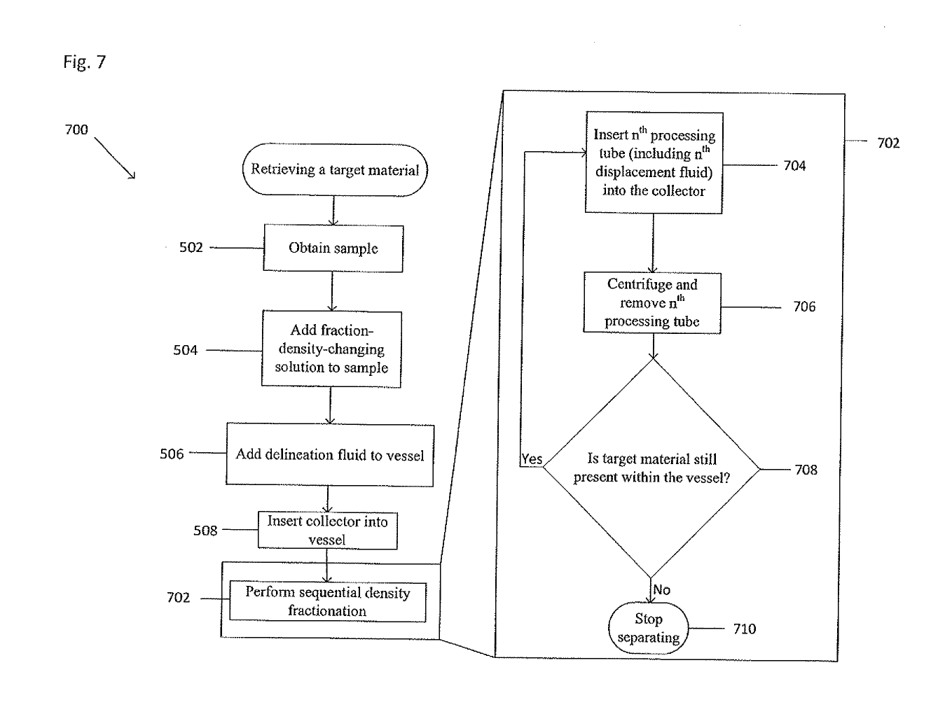

FIG. 7 shows an example method 700 for retrieving a target material using sequential density fractionation. The example method 700 is similar to the example method 500, except the example method 700 collects the target material by sequential density fractionation. After the collector has been inserted into the vessel, as shown in block 508, sequential density fractionation is performed, as seen in block 702. Block 702 is also a snapshot of the sequential density fractionation steps. In block 704, an n^{th} processing receptacle including an n^{th} displacement fluid is inserted into the collector, such that n^{th} is greater than or equal to first (i.e. second, third, fourth, and so on). In block 706, the system is centrifuged to collect a fraction or sub-fraction and the nth processing receptacle is removed. In block 708, the operator determines whether or not the desired fraction or sub-fraction is obtained. When the desired fraction or sub-fraction is obtained, the process may stop as shown in block 710, though the process may continue until all fractions or sub-fractions are obtained. When the desired fraction or sub-fractions is not yet obtained, the process restarts at block 704. The processing receptacles may also include a processing solution to effect a change on the respective sub-fractions. Two or more processing receptacles and respective displacement fluids may be used depending on the number of fractions or sub-fractions desired for separation and collection. Each successive displacement fluid is denser than the preceding displacement fluid. Similarly, each successive fraction or sub-fraction is denser than the preceding fraction or sub-fraction. Once collected, the consecutive sub-fractions may be analyzed, such as for diagnostic, prognostic, research purposes, to determine components characteristics (i.e. a complete blood count), how those characteristics change over time, or the like.

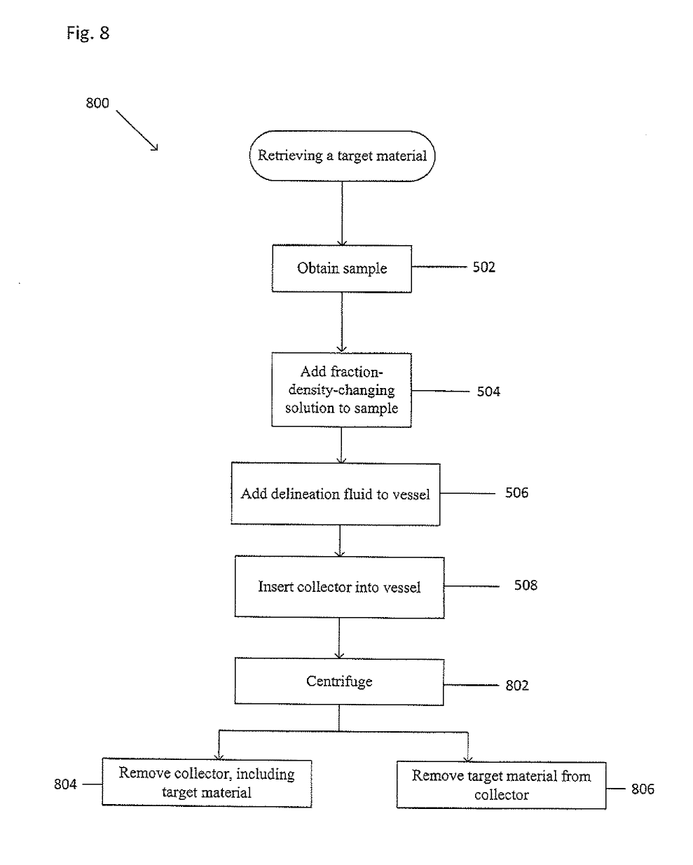

FIG. 8 shows an example method 800 for retrieving a target material. The example method 800 is similar to the example method 500, except the example method 800 does not use a processing vessel, as the target material may be collected in the collector. After the collector has been inserted into the vessel, as seen in block 508, the vessel and collector are centrifuged, as seen in block 802. In block 804, the collector, including the target material, may be removed from the vessel. Alternatively, in block 806, the target material collected within the collector may be removed from the collector with a processing receptacle, such as a tube, a pipet, a needle, a syringe, or the like. An appropriate amount of delineation fluid may have been added to the vessel prior to centrifugation to lift the target material into the collector. Additionally, a displacement fluid may be added to the vessel prior to or before the collector to lift the target material into the collector.

The target material may be analyzed using any appropriate analysis method or technique, though more specifically extracellular and intracellular analysis including intracellular protein labeling; chromogenic staining; molecular analysis; genomic analysis or nucleic acid analysis, including, but not limited to, genomic sequencing, DNA arrays, expression arrays, protein arrays, and DNA hybridization arrays; in situ hybridization (“ISH”—a tool for analyzing DNA and/or RNA, such as gene copy number changes); polymerase chain reaction (“PCR”); reverse transcription PCR; or branched DNA (“bDNA”—a tool for analyzing DNA and/or RNA, such as mRNA expression levels) analysis. These techniques may require fixation, permeabilization, and isolation of the target material prior to analysis. Some of the intracellular proteins which may be labeled include, but are not limited to, cytokeratin (“CK”), actin, Arp2/3, coronin, dystrophin, FtsZ, myosin, spectrin, tubulin, collagen, cathepsin D, ALDH, PBGD, Akt1, Akt2, c-myc, caspases, survivin, p27^{kip}, FOXC2, BRAF, Phospho-Akt1 and 2, Phospho-Erk1/2, Erk1/2, P38 MAPK, Vimentin, ER, PgR, PI3K, pFAK, KRAS, ALKH1, Twist1, Snail1, ZEB1, Fibronectin, Slug, Ki-67, M30, MAGEA3, phosphorylated receptor kinases, modified histones, chromatin-associated proteins, and MAGE. To fix, permeabilize, or label, fixing agents (such as formaldehyde, formalin, methanol, acetone, paraformaldehyde, or glutaraldehyde), detergents (such as saponin, polyoxyethylene, digitonin, octyl β-glucoside, octyl β-thioglucoside, 1-S-octyl-β-D-thioglucopyranoside, polysorbate-20, CHAPS, CHAPSO, (1,1,3,3-Tetramethylbutyl)phenyl-polyethylene glycol or octylphenol ethylene oxide), or labeling agents (such as fluorescently-labeled antibodies, enzyme-conjugated antibodies, Pap stain, Giemsa stain, or hematoxylin and eosin stain) may be used.

After collection, the target material may also be imaged or may undergo flow cytometry. To be imaged, a solution containing a fluorescent probe may be used to label the target material, thereby providing a fluorescent signal for identification and characterization, such as through imaging. The solution containing the fluorescent probe may be added to the suspension before the suspension is added to the vessel, after the suspension is added to the vessel but before centrifugation, or after the suspension has undergone centrifugation. The fluorescent probe includes a fluorescent molecule bound to a ligand. The target material may have a number of different types of surface markers. Each type of surface marker is a molecule, such an antigen, capable of attaching a particular ligand, such as an antibody. As a result, ligands may be used to classify the target material and determine the specific type of target materials present in the suspension by conjugating ligands that attach to particular surface markers with a particular fluorescent molecule. Examples of suitable fluorescent molecules include, but are not limited to, quantum dots; commercially available dyes, such as fluorescein, Hoechst, FITC (“fluorescein isothiocyanate”), R-phycoerythrin (“PE”), Texas Red, allophycocyanin, Cy5, Cy7, cascade blue, DAPI (“4′,6-diamidino-2-phenylindole”) and TRITC (“tetramethylrhodamine isothiocyanate”); combinations of dyes, such as CY5PE, CY7APC, and CY7PE; and synthesized molecules, such as self-assembling nucleic acid structures. Many solutions may be used, such that each solution includes a different type of fluorescent molecule bound to a different ligand.

The following is an example method for retrieving a target material when the density of the plasma is changed to be greater than the density of the buffy coat but less than the density of the red blood cells:
1. Determine hematocrit or packed cell volume (“PCV”).
2. Combine 4 mL of blood (42% PCV) with 8 mL of 1.12 g/mL Percoll in tube (to change plasma density to approximately 1.1 g/mL).
3. Layer 1.085 g/mL Percoll on top within tube.
4. Insert collector with waste vessel into tube.
5. Remove waste vessel.
6. Insert processing receptacle with 1.0606 g/mL Percoll into collector.
7. Centrifuge 1000G for 30 minutes.
8. Remove processing receptacle with sub-fraction of buffy coat having a density less than 1.0606 g/mL.
9. Insert processing receptacle with 1.0757 g/mL Percoll into collector.
10. Centrifuge 1000G for 30 minutes.
11. Remove processing receptacle with sub-fraction of buffy coat having a density between 1.0606 g/mL and 1.0757 g/mL.
12. Insert processing receptacle with 1.00770 g/mL Percoll into collector.
13. Centrifuge 1000G for 30 minutes.
14. Remove processing receptacle with sub-fraction of buffy coat having a density between 1.0757 g/mL and 1.0770 g/mL.

The following is an example method for retrieving a target material when the density of the plasma is changed to be greater than the densities of both the buffy coat and the red blood cells:
1. Combine 4 mL of blood with 2 mL of 1.3 g/mL OptiPrep in tube (to change plasma density to be greater than the density of the red blood cells).
2. Layer 1.085 or 1.1 g/mL Percoll on top within tube.
3. Insert collector with waste vessel into tube.
4. Remove waste vessel.
5. Insert processing receptacle with 1.0606 g/mL Percoll into collector.
6. Centrifuge 1000G for 30 minutes.
7. Remove processing receptacle with sub-fraction of buffy coat having a density less than 1.0606 g/mL.
8. Insert processing receptacle with 1.0757 g/mL Percoll into collector.
9. Centrifuge 1000G for 30 minutes.
10. Remove processing receptacle with sub-fraction of buffy coat having a density between 1.0606 g/mL and 1.0757 g/mL.
11. Insert processing receptacle with 1.00770 g/mL Percoll into collector.
12. Centrifuge 1000G for 30 minutes.
13. Remove processing receptacle with sub-fraction of buffy coat having a density between 1.0757 g/mL and 1.0770 g/mL.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents:

I/We claim:

1. A method for collecting a target material from a sample, the method comprising the steps of:
adding a fraction-density-altering solution to a vessel that contains the sample, the fraction-density-altering solution to change the density of a first fraction of the sample such that the density of the target material is less than a changed density of the first fraction;
inserting a collector that includes a first processing receptacle into an open end of the vessel; and
centrifuging the vessel and the collection system to force at least a first portion of the target material into the first processing receptacle.

2. The method of claim 1, the collector comprising:
a main body including a bore extending through the main body.

3. The method of claim 1, the collector comprising:
a primary body including a first end and an opposing second end, the primary body including:
a conical-shaped opening in the second end that narrows to an apex within the primary body, and
a cavity with an opening at the first end; and
a cannula that extends from the apex into the cavity, the cannula to provide an opening between the conical-shaped opening and the cavity.

4. The method of claim 1, wherein the fraction-density-solution is selected from the group consisting of a solution of colloidal silica particles coated with polyvinylpyrrolidone, a polysaccharide solution, iodixanol, a complex, branch glucan, cesium chloride, sucrose, a sugar-based solution, a polymer solution, and a multi-phase polymer solution.

5. The method of claim 1, further comprising:
adding a delineation fluid having a density greater than the target material and less than the changed density of the first sample fraction and the density of any other sample fractions to the vessel prior to inserting the collector into the vessel.

6. The method of claim 5, further comprising adding an insert to the vessel prior to adding the delineation fluid to control the addition of the delineation fluid and inhibit mixing with the sample and the fraction-density-altering solution.

7. The method of claim 1, further comprising:
adding a delineation fluid having a density greater than the target material and less than the changed density of the first sample fraction and the density of any other sample fractions to the vessel through the collector after inserting the collection system into the vessel.

8. The method of claim 1, wherein the first processing receptacle includes a first displacement fluid having a density greater than the target material and less than the changed density of the first sample fraction and any other sample fractions,
the first displacement fluid to flow into the vessel via the collector and the target material to flow into the first processing receptacle via the collector during the centrifuging step.

9. The method of claim 1, wherein the first processing receptacle includes a first displacement fluid having a density greater than a first sub-fraction of the target material and less than the changed density of the first sample fraction and any other sample fractions,
the first displacement fluid to flow into the vessel via the collector and the first sub-fraction of the target material to flow into the first processing receptacle via the collector during the centrifuging step.

10. The method of claim 9, further comprising the steps of:
removing the first processing receptacle including the first sub-fraction from the collector;
inserting a second processing receptacle including a second displacement fluid into the chamber of the collector; and
re-centrifuging the primary vessel with the collector and the second processing vessel,
the second displacement fluid to flow into the primary vessel via the collector and a second sub-fraction to flow into the second processing receptacle via the collector,
wherein the second displacement fluid has a density greater than the second sub-fraction and the first displacement fluid, and wherein the second sub-fraction has a density greater than the first sub-fraction.

11. The method of claim 10, wherein the first sub-fraction is the target material.

12. The method of claim 10, wherein the first sub-fraction is a non-target material and the second sub-fraction is the target material.

13. The method of claim 10, wherein the removing, inserting, and re-centrifuging steps are repeated with an n^{th} processing receptacle including an n^{th} displacement fluid, wherein n^{th} is equal to or greater than and wherein the removing, inserting, and re-centrifuging steps are repeated until all desired sub-fractions are obtained from the sample, wherein each successive displacement fluid has a density greater than each preceding displacement fluid.

14. The method of claim 1, wherein the vessel includes a septum in a closed end.

15. The method of claim 14, further comprising removing the first sample fraction from the vessel via the septum after the centrifuging step.

16. The method of claim 1, further comprising:
centrifuging the vessel that contains the sample to separate the sample intro fractions; and
removing at least a portion of the first sample fraction,
wherein these steps are performed before adding the fraction-density-altering solution.

17. The method of claim 1, wherein the fraction-density-altering solution does not change the density of the target material.

18. The method of claim 17, wherein the fraction-density-altering solution does not change the density of any other fraction of the sample.

19. A method for collecting a target material from a sample, the method comprising the steps of:
adding a fraction-density-altering solution to a vessel that contains the sample, the fraction-density-altering solution to change the density of a first fraction of the sample such that the density of the target material is less than a changed density of the first fraction;
inserting a collector into an open end of the vessel; and
centrifuging the vessel and the collector to force at least a first portion of the target material from the vessel and into the collector.

20. The method of claim 19, the collector comprising:
a main body including a bore extending through the main body.

21. The method of claim 19, the collector comprising:
a primary body including a first end and an opposing second end, the primary body including:
a conical-shaped opening in the second end that narrows to an apex within the primary body, and
a cavity with an opening at the first end; and
a cannula that extends from the apex into the cavity, the cannula to provide an opening between the conical-shaped opening and the cavity.

22. The method of claim 19, further comprising the step of removing the collector from the vessel after the centrifuging step.

23. The method of claim 19, further comprising the step of removing the target material from the collector with a processing receptacle.
